(19) United States
(12) Patent Application Publication
(10) Pub. No.: US 2003/0124009 A1
(43) Pub. Date: Jul. 3, 2003

(54) Hydrophilic polymer actuators

(76) Inventors: Vilupanur A. Ravi, Claremont, CA (US); Morteza Gharib, San Marino, CA (US); Benjamin A. Welander, Monmouth, OR (US)

Correspondence Address: CHRISTIE, PARKER & HALE, LLP, P.O. BOX 7068, PASADENA, CA, 91109-7068, US

(21) Appl. No.: 10/280,140

(22) Filed: Oct. 23, 2002

Related U.S. Application Data
(60) Provisional application No. 60/335,206, filed on Oct. 23, 2001.

(51) Int. Cl. A61F 2/08

(52) U.S. Cl. 417/572; 91/471; 623/14.13; 73/862.381

(57) ABSTRACT

Hydrophilic polymer actuators for an implantable device and methods of forming such actuators are provided, wherein the hydrophilic polymer actuators are actuated by the hydration and dehydration of a hydrophilic polymer material.

### CROSS-REFERENCE TO RELATED APPLICATIONS

This application claims priority based on U.S. provisional application No. 60/335,206, filed Oct. 23, 2001, the disclosure of which is incorporated herein by reference.

### FIELD OF INVENTION

The invention relates to polymeric actuators, and more particularly to hydrophilic polymeric actuators.

### BACKGROUND OF INVENTION

There has been a great deal of recent interest in developing new, synthetic, biomedical devices for use in replacing defective tissues and organs in patients. Although, a great deal of progress has been made in this field, many of the devices of interest would require motors and actuators to operate that continue to have serious performance deficiencies.

The development of artificial muscles is a case in point. Current artificial actuator and motor technologies can provide stresses, percent deformation, cycle rates or efficiencies comparable to biological muscle; but so far no single device meets all of the necessary performance criteria.

To resolve these deficiencies a number of different methods of actuation have been proposed to activate different types of materials, such as, for example, changes in electric fields, magnetic fields, light, pH, temperature, and even body chemistry. Some examples are provided by Y. Bar-Cohen (Proc. AIAA, vol. 2001-1492 (Apr. 16-19, 2001)) and L. Liang, et al. (Langmuir, vol. 16, pg. 8016 (2000)), the disclosures of which are incorporated herein by reference.

Recent research by Pelrine, et al. (Science, vol. 287, pg. 836 (2001)) indicates that using an electrostriction actuator provides the closest performance to natural muscle. Polymers exhibiting this property, however, require large electric voltages (˜412 kV/mm) for activation, resulting in a potentially hazardous discharge within the body.

Accordingly, a need exists for a safe, reliable actuator capable of providing stresses, percent deformation, cycle rates and efficiencies comparable to biological muscle.

### SUMMARY OF THE INVENTION

The present invention is directed to hydrophilic polymer actuators for an implantable device that are actuated by hydration and dehydration of the polymer material.

In one exemplary embodiment of the invention, the hydrophilic polymer actuators, in addition to other criteria, would be capable of producing forces of about 0.1 N (˜10 gf) corresponding to stresses in the range of 0.15˜0.3 MPa and cycle times of about 1 sec.

In another exemplary embodiment of the invention, the hydrophilic polymer materials are in either monolithic or coated form.

In still another exemplary embodiment of the invention, the polymer materials are polyurethanes. In such an embodiment the polyurethane polymer material may be formed by one of, or a combination of, pre-stressed, and thinner films, to provide reduced cycle times.

In yet another exemplary embodiment of the invention, hydrogel-coated membranes may be used as the polymer materials.

In still yet another exemplary embodiment of the invention, the hydrophilic polymer actuators may also be utilized as devices for microfluid transport.

In still yet another exemplary embodiment, the invention is directed to a method of forming a hydrophilic polymer actuator in accordance with the invention.

In still yet another exemplary embodiment, the invention is directed to devices for measuring the properties of the hydrophilic polymer actuators in accordance with the invention.

### BRIEF DESCRIPTION OF THE DRAWINGS

These and other features and advantages of the present invention will be better understood by reference to the following detailed description when considered in conjunction with the accompanying drawings wherein:

FIG. 1 is a schematic diagram of a hydrophilic polymeric actuator in accordance with an exemplary embodiment of the invention.

FIG. 2 is a schematic diagram of a hydrophilic polymeric actuator during actuation in accordance with an exemplary embodiment of the invention.

FIG. 3 is a side view of an exemplary embodiment of an apparatus for measuring the properties of the hydrophilic polymeric actuators of the current invention.

FIG. 4 is a side view of an exemplary embodiment of an apparatus for measuring the properties of the hydrophilic polymeric actuators of the current invention.

FIG. 5 is a side view of an exemplary embodiment of an apparatus for measuring the properties of the hydrophilic polymeric actuators of the current invention.

FIG. 6 is a graphical depiction of the mechanical response of an exemplary embodiment of the hydrophilic polymeric actuators in accordance with present invention.

FIG. 7 is a graphical depiction of the strain response of an exemplary embodiment of the hydrophilic polymeric actuators in accordance with present invention.

FIG. 8 is a graphical depiction of the wetting response of an exemplary embodiment of the hydrophilic polymeric actuators in accordance with present invention.

FIG. 9 is a graphical depiction of the swell/shrink cycle response of an exemplary embodiment of the hydrophilic polymeric actuators in accordance with present invention.

FIG. 10 is a graphical depiction of the cycle time of exemplary embodiments of the hydrophilic polymeric actuators in accordance with present invention.

FIG. 11 is a graphical depiction of the wetting response of an exemplary embodiment of the hydrophilic polymeric actuators in accordance with present invention.

### DETAILED DESCRIPTION OF THE INVENTION

The present invention is directed to hydrophilic polymer actuators for an implantable device that are actuated by the hydration and dehydration of a hydrophilic polymer material.

It has long been known that hydrophilic polymers, such as polyurethanes, can undergo significant volume changes upon exposure to water and subsequent drying. Applicants have discovered that the swelling and shrinking forces of these hydrophilic polymer materials in response to liquids are capable of serving as an activation or actuation mechanism for a device.

Specifically, Applicants have discovered that by proper selection of the material and the actuator geometry, and by careful control over the wetting of the polymer material, forces of about 0.1 N can be generated with response frequencies approaching 1 Hz using the swelling and shrinking of these hydrophilic polymer materials. For example, the magnitude of the swelling forces (and corresponding stresses) produced in the hydrophilic polymer materials of the current invention increase with the amount of water added to the polymer (or the area of polymer wetted). However, the cycle times (time differentials between points where a drop in force changes to an increase in force) of the hydrophilic polymer actuators also increase with increased wetting. Accordingly, it is necessary to carefully balance the wetting to the actuator to ensure that both the generated force and cycle time of the actuator are optimized.

However, cycle time and generated force can also be influence by the design of the actuator itself. For example, thinner polyurethanes provide faster cycle times, such that by varying the thickness, the amount of water, and the extent of area covered during hydration the force-time response of these polyurethane actuators may be tailored for each specific application.

Although only the use of polyurethanes is discussed above, it should be understood that any hydrophilic polymer capable of producing the requisite forces and cycle times through liquid induced swelling and shrinkage may be used in the current invention. For example, polymer actuators made with hydrogel coatings are also capable of meeting these requirements. In addition, hydrogel coatings of different chemistries and thicknesses may be applied to different substrates to obtain a force-time response with respect to wetting and drying tailored for a particular application.

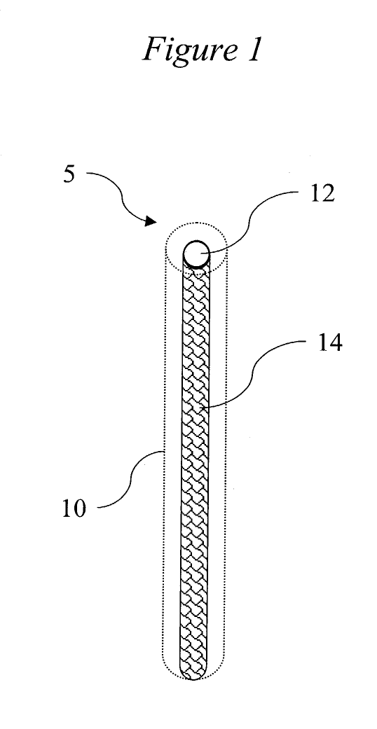

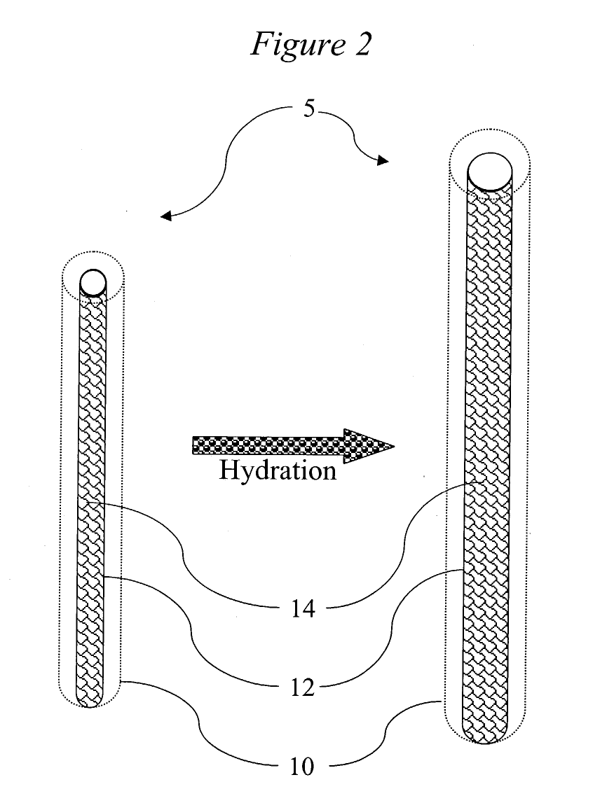

One exemplary embodiment of a hydrophilic polymeric actuator in accordance with the current invention is provided in FIGS. 1 and 2. As shown, the actuator 5 comprises two coaxial cylinders 10 and 12 having diameter of about 2 mm. Although the cylinders 10 and 12 may be made of any suitable hydrophilic polymer, in the embodiment shown in FIGS. 1 and 2, the cylinders are made of a hydrophilic polyurethane polymer material 14, which has a volume displacement of about 15% when wet and is capable of generating a force of about 0.1 N with a response time of about 1 Hz.

As shown in FIG. 2, during operation wetting an area of the polymer causes the polymer in the wet area to swell, which in turn causes macroscopic movement of the actuator. This movement and the force exerted during this movement can be utilized for a number of applications, such as, for example, movement, actuation, and manipulation of other objects. In addition, utilizing the coaxial design shown, can provide a pumping action that would allow the actuator to move liquids in a controlled fashion.

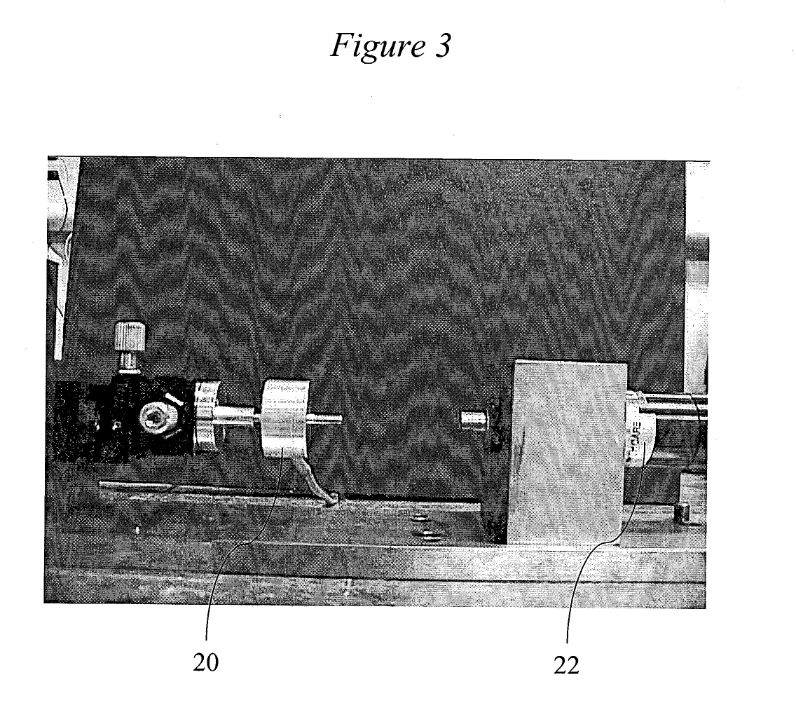

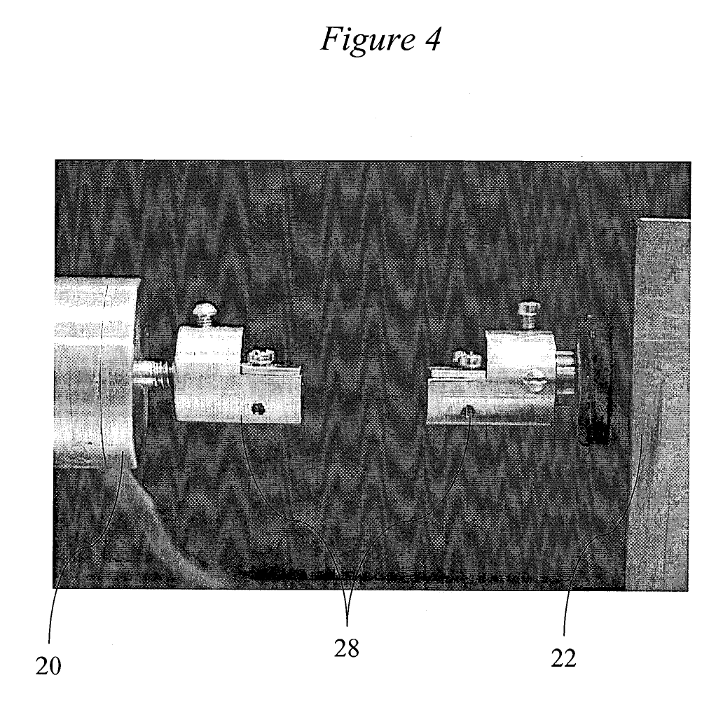

The invention is also directed to a method of measuring the force generated by the actuators of the current invention. One exemplary embodiment of an apparatus for conducting swelling and shrinking force measurements is shown in FIGS. 3 to 5. As shown in FIG. 3, the measurement apparatus consists of a load cell 20 with a 1 mg resolution attached opposite a mechanical inch worm micrometer 22. The micrometer can move at varying speeds on the order of about 1 μm/sec. Both the micrometer 22 and load cell 20 are connected to measurement and control electronics (not shown) installed in a PC (not shown) provided with a program capable of controlling the micrometer and recording force data. As shown in FIG. 4, grips 28, comprising in this embodiment two plates and hold screws, are provided to attach to the load cell 20 and micrometer 22.

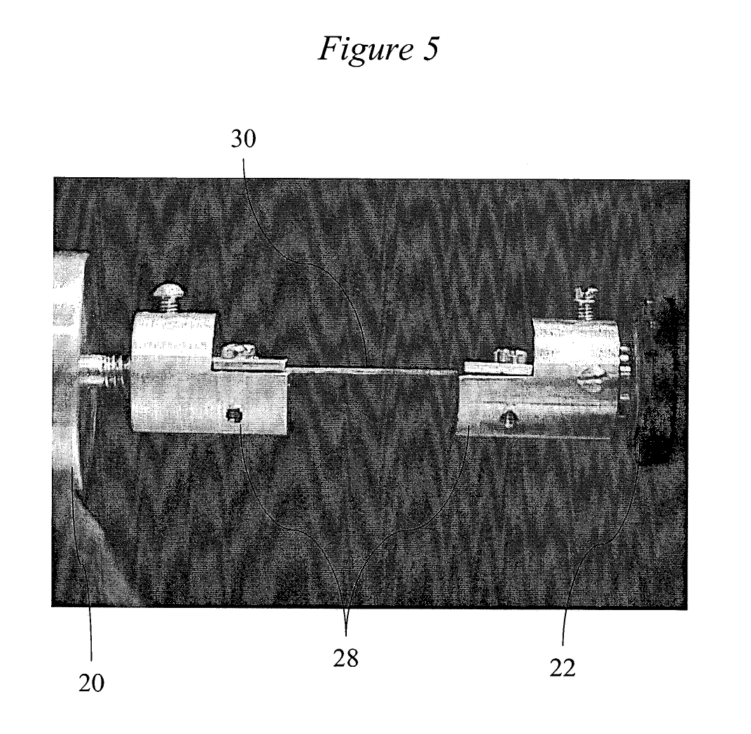

During a measurement, as shown in FIG. 5, each end of the sample 30 is firmly gripped between the plates of the grips 28. The plates are designed to be deep and wide enough to prevent the screws from touching the sample 30. Once the test sample is secured in place by tightening the screws of the grips, the separation distance between the load cell and the micrometer is adjusted so that the sample is taut with a nominal tensile loading of 0.5±0.3 gram force (gf). The samples are then pre-strained to 1.3% (0.2+/−0.01 mm) by pulling the grips apart at a rate of 1.1 μm/sec. A standard strain can be determined by stretching trial samples and finding the strain which produces the maximum force that can be attained without damaging the load cell. A high pre-stress can be used to obtain the maximum force change when the sample is wet and swollen.

Force data may be collected at any suitable time interval (such as, 5-second intervals for standard measurements and 1 second intervals for higher resolution measurements) and later transferred to a computer for analysis. During dry tests, the sample should not be disturbed for at least 4500 seconds so that the deformation can be studied by measuring force as a function of time. During wet tests, the sample may be moistened using a variety of methods, such as, for example, by syringe, pipette, or cosmetics sponge. The choice of the method of application depends solely upon the extent and type of wetting being investigated.

### EXAMPLE

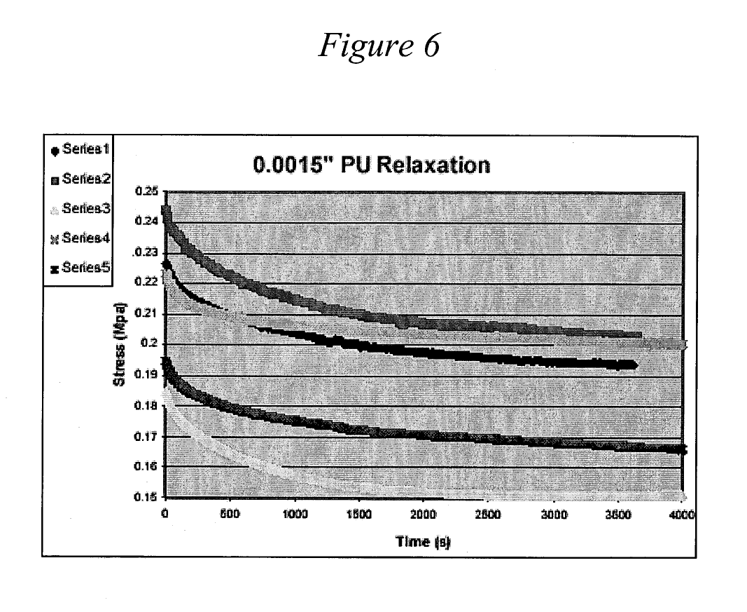

FIGS. 6 to 11 show the results of force tests conducted by Applicants on exemplary embodiments of actuators in accordance with the current invention. For these tests, polyurethane samples were obtained from Thermedics Polymer Products, Inc (Woburn, Mass.) as rolls 2″ wide and 0.015″ thick, and as sheets 0.010″ and 0.005″ thick from Specialty Extrusion (Fullerton, Calif.). Hydrogel samples were obtained from Hydromer, Inc. (Somerville, N.J.).

FIG. 6 shows the mechanical response of dry polyurethane over time after a pre-strain of 1.3%. After data acquisition commenced, the samples were allowed to relax. It should be noted that sibling samples behaved similarly and that for any sample, the measured drop in stress did not exceed 0.04 Mpa, showing that absent a hydration/dehydration response there is little force generated by the polymer actuators. Small perturbations in the curves shown in FIG. 6 may have been caused by disturbances of the air within the test area.

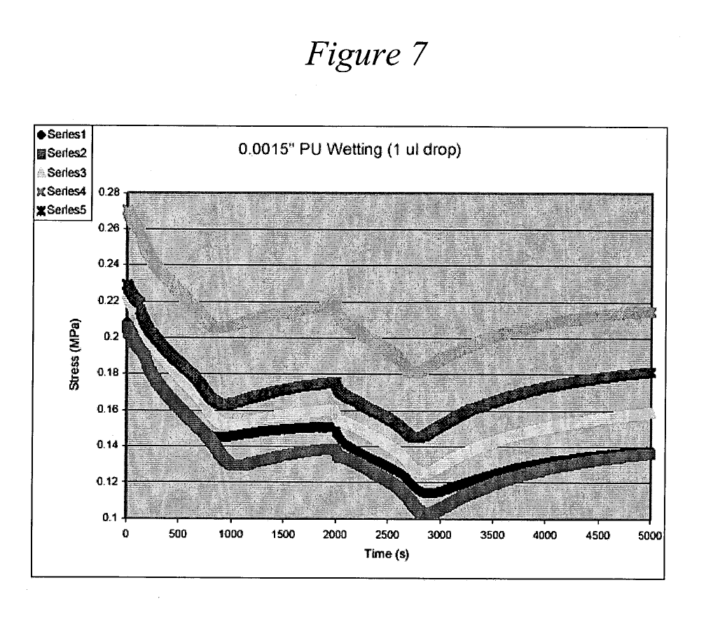

FIG. 7 shows the effect of placing a 1 μl drop of water on a sample of polyurethane after being strained to 1.3%. A drop was placed on each sample at 120 seconds and at 2000 seconds. After the first drop of water was applied, each of the samples showed a decline in stress of at least 0.05 MPa followed by a recovery in stress of about 0.01 MPa. After the second water drop, the samples released about 0.04 Mpa of stress. Note that all but one sample recovered the loss of stress due to the second drop by about the 5000-second mark showing the force generated by the polymer actuators of the present invention as a result of a hydration/dehydration cycle.

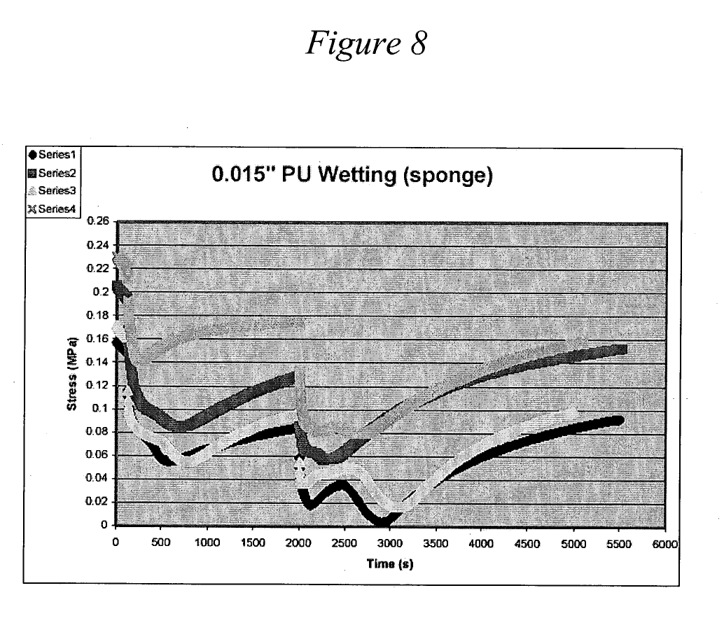

FIG. 8 shows the effect of wetting the entire side of each sample using a cosmetics sponge. Samples were wetted at 120 and 2000 seconds. The decline in stress caused by the first wetting ranges from about 0.08 to 0.11 Mpa, depending upon the amount of water delivered to the sample by the sponge. The second wetting produced decreases in stress of between about 0.06 to 0.09 MPa. Note that even this large stress was recovered by three out of four samples by the 5000-second mark. The variation in the force generated by these samples is believed to be the result of the relatively uncontrolled method of wetting. For example, instead of a single controlled drop, or even an even film of water being deposited on the surface of the sample, the water coalesced into small drops. Different size drops left behind by the sponge produced varying shapes of the minimums, and the size and number of these small drops affected the position of the minimum during the test.

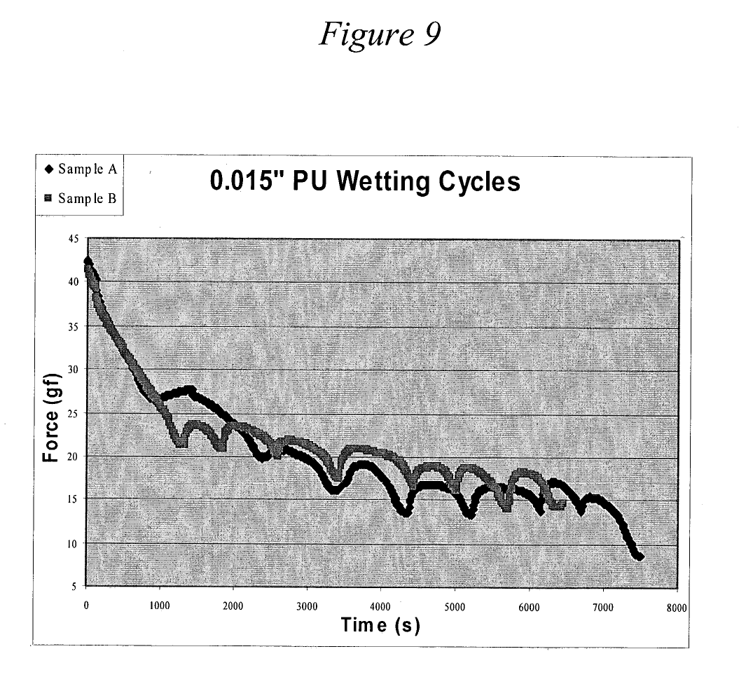

FIG. 9 graphically illustrates two exemplary embodiments of actuator system designs to increase the swelling/shrinking cycle frequency of the actuators according to the current invention.

For example, Sample A was wet repeatedly in order to release as much tension as possible using only μl drops. During this test, at about 3600 seconds, a drop was added and the force increased. The same phenomenon was observed for five consecutive wettings. A second trial was run to make sure this “jump” effect was not a peculiarity of this sample. Using Sample B, sharp valleys were created by placing a drop at the lowest point of force, and again each drop of water caused the tensile force to increase. As the drop was absorbed into the sample, however, the force began to decrease. When the decline in tension appeared fastest, another drop was added and the process repeated. The amplitudes of each cycle are on the order of 3 to 4 gf. Accordingly, by saturating the actuator material it is possible to increase the frequency of the force response.

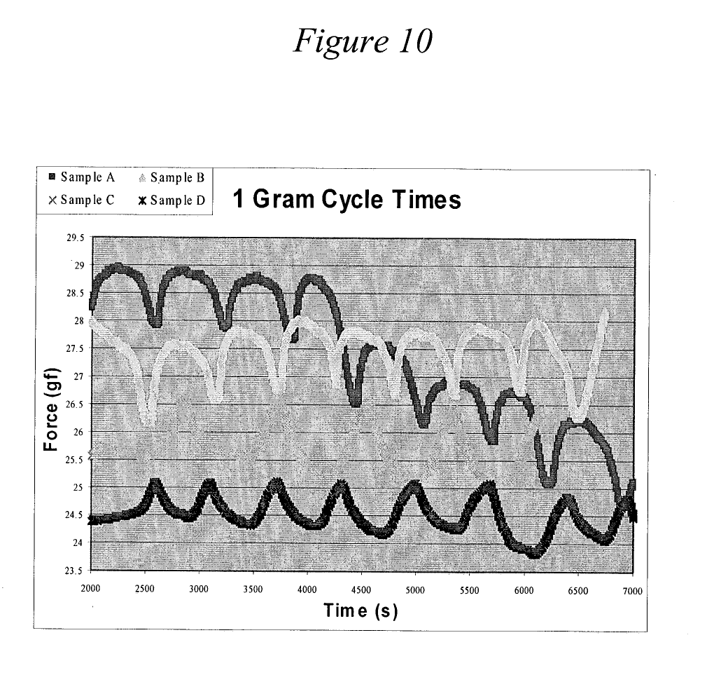

FIG. 10 displays the cycle time needed to achieve 1 gf using samples of different thickness. The legend shows the thickness of the sample and the size of the drop of water used. An arbitrary force of 13 gf was added to each force datum of the 5 mil (0.005″) samples to provide clarity to the graph. The time scale begins at 2000 s because this “preconditioning” time was needed to drop the force from the initial maximum at pre-strain to an appropriate level. At times less than 2000 seconds, the tension was usually in a constant decline from the initial strain. Samples A, B and C used 600 nl drops of water and Sample D used 400 nl drops. Sample A was 0.015″ thick and took advantage of the jump mechanism described earlier to produced a change in tension of 1 gf about every 630 seconds. The 0.010″ sample, B, did the same in about 600 seconds. Sample C, 0.005″ thick, did not respond similarly because the increase in force was always less than 1 gf before a drop in the tensile forces. Therefore, the slower mechanism of absorption-evaporation was used instead to produce cycle times of 800 seconds. By reducing the amount of water to 400 nl drops, as in Sample D, the time was cut to approximately 675 seconds. Accordingly, these results show that by proper selection of actuator geometry, thickness and volume of liquid, the force generated and cycle time may be controlled.

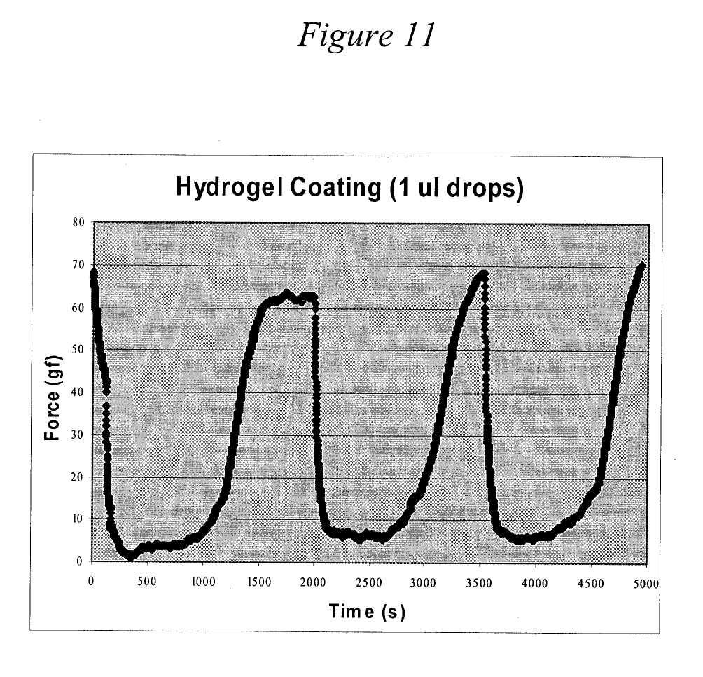

Although the previous Figures show the results from test involving polyurethane samples, FIG. 11 shows the wetting of a hydrophobic membrane coated with a hydrogel. As shown, a large initial force developed after only a 0.26% stain. The initial decrease to ˜44 gf was due to the sample relaxing. The steeper slope and all subsequent drops in force were caused by the addition of 1 μl of water. The release of tension due to the first drop was about 43 gf The drops in force due to the second and third drops are ˜54 gf and 69 gf, respectively. The recovery after the first drop seemed to peak around 63 gf. Subsequent recoveries were interrupted to keep the tensile stress within safe operating limits of the load cell.

It should be noted that for all of the samples, for initial comparisons, all samples were wetted 120 and 2000 seconds after initial straining by placing a 1 μl drop in the center of the sample. The sponge was later used to wet the entire sample to examine the effect of the amount of water added, and sample area wetted.

From these tests, Applicants determined that:

The force generated by swelling and shrinking is nominally the same for sibling polymers, but decreases as the thickness of the sample decreases.

By using thinner film samples, and by increasing the pre-strain, the absorption/evaporation cycle times may be decreased. In addition, reducing the volume of water placed on the sample also enables one to controllably reduce the cycle time of the actuator.

The “jump” phenomenon associated with depositing large amounts of water appears to be a faster mechanism for force change than absorption/evaporation. Although not to be bound by theory, it is believed that the cohesive forces of water are enough to pull moisture back out of the polymer after it has reached a certain saturation level. When the amount of moisture in the area of the polymer directly adjacent to the drop of water has decreased, either by diffusion or evaporation, below this threshold, the polymer will begin to absorb the drop.

Finally, although polyurethanes are chiefly discussed above, the response of hydrogel-coated films appears to meet the force criterion of the actuators of the current invention, while the cycle times for this material is shorter. Further by making the thicknesses of the hydrophilic polymers smaller, faster cycle times can be obtained.

The preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention.

Accordingly, the foregoing description should not be read as pertaining only to the precise structures described and illustrated in the accompanying drawings, but rather should be read consistent with and as support to the following claims which are to have their fullest and fair scope.

What is claimed is:

1. A polymeric actuator comprising:
a hydrophilic polymeric material capable of generating an actuation force responsive to the hydration and dehydration of the hydrophilic polymeric material within an actuation cycle time.

2. The polymeric actuator described in claim 1, wherein the hydrophilic polymeric material is a polyurethane.

3. The polymeric actuator described in claim 1, wherein the hydrophilic polymeric material is further coated with a hydrogel material.

4. The polymeric actuator described in claim 1, wherein the actuation force is at least about 0.1N.

5. The polymeric actuator described in claim 1, wherein the actuator produces a stress in the range of about 0.15-0.3 MPa

6. The polymeric actuator described in claim 1, wherein the polymeric material is in the form of one of either a monolith or coated polymer material.

7. The polymeric actuator described in claim 1, wherein the polymeric material is pre-stressed.

8. The polymeric actuator described in claim 1, wherein the polymeric material is in the form of a strip.

9. The polymeric actuator described in claim 1, wherein the polymeric material is in the form of at least one cylinder.

10. The polymeric actuator described in claim 1, wherein the polymeric material is in the form of two coaxial cylinders.

11. The polymeric actuator described in claim 1, wherein the actuator is designed as a fluid pump.

12. The polymeric actuator described in claim 1, wherein the actuation cycle time is about 1 Hz.

13. The polymeric actuator described in claim 1, wherein the actuation cycle time is dependent on the thickness of the polymeric material and wherein the polymeric material has a thickness of less than about 0.015 inches.

14. A method of forming a polymeric actuator comprising the steps of:
providing a hydrophilic polymeric material capable of generating an actuation force responsive to the hydration and dehydration of the hydrophilic polymeric material within an actuation cycle time; and
providing a source of hydration fluid in fluid communication with the hydrophilic polymeric material.

15. The method described in claim 14, wherein the hydrophilic polymeric material is a polyurethane.

16. The method described in claim 14, wherein the hydrophilic polymeric material is further coated with a hydrogel material.

17. The method described in claim 14, wherein the actuation force is at least about 0.1 N.

18. The method described in claim 14, wherein the polymeric material is in the form of one of either a monolith or coated polymer material.

19. The method described in claim 14, further including the step of pre-stressing the polymeric material.

20. The method described in claim 14, wherein the step of providing the polymeric material further includes forming the polymeric material into a strip.

21. The method described in claim 14, wherein the step of providing the polymeric material further includes forming the polymeric material into a cylinder.

22. The method described in claim 14, wherein the step of providing the polymeric material further includes forming the polymeric material into two coaxial cylinders.

23. The method described in claim 14, wherein the actuation cycle time is about 1 Hz.

24. The method described in claim 14, wherein the actuation cycle time is dependent on the thickness of the polymeric material, and wherein the polymeric material has a thickness of less than about 0.015 inches.

25. The method described in claim 14, further comprising the step of exposing the hydrophilic polymeric material to a hydration volume the hydration fluid at a hydration rate such that the hydrophilic polymeric material generates the actuation force.

26. The method described in claim 25, further comprising the step of controlling the hydration volume, wherein the actuation cycle time is dependent on the hydration volume.

27. The method described in claim 26, wherein the step of controlling the hydration volume includes hydrating the polymeric material to a saturation level such that cohesive forces of the hydrating fluid are sufficient to remove hydration fluid from the polymeric material.

28. A polymeric actuator force testing apparatus comprising:
a load cell;
a moveable stage aligned opposite the load cell and capable of moving along an axis toward and away from the load cell;
a hydration fluid source positioned above the axis between the load cell and moveable stage; and
wherein a polymeric sample may be mounted along the axis between load cell and the moveable stage such that the moveable stage may apply a pre-stress to the polymeric sample, and such that a force generated by the polymeric sample is measured by the load cell.

29. A method of measuring the force generated by a polymeric sample comprising the steps of:
providing a load cell;
providing a moveable stage aligned opposite the load cell and capable of moving along an axis toward and away from the load cell;
providing a hydration fluid source positioned above the axis between the load cell and moveable stage;
mounting a polymeric sample along the axis between load cell and the moving stage;
applying a pre-stress to the polymeric sample by moving the moveable stage away from the load cell;
hydrating the polymeric sample from the hydration source; and
measuring the force generated by the polymeric sample on the load cell.
